(19) United States
(12) Patent Application Publication
(10) Pub. No.: US 2009/0012023 A1
(43) Pub. Date: Jan. 8, 2009

(54) DECOY-OLIGNUCLEOTIDE INHIBITION OF CD40-EXPRESSION

(76) Inventors: Markus Hecker, Gottingen (DE); Andreas H. Wagner, Gottingen (DE)

Correspondence Address: FULBRIGHT & JAWORSKI L.L.P., 600 CONGRESS AVE., SUITE 2400, AUSTIN, TX, 78701, US

(21) Appl. No.: 11/961,621

(22) Filed: Dec. 20, 2007

Related U.S. Application Data
(62) Division of application No. 10/526,521, filed on Mar. 1, 2005.

(30) Foreign Application Priority Data
Sep. 2, 2002 (DE) 102 40 417.8

(51) Int. Cl. A61K 31/713; C07H 21/04

(52) U.S. Cl. 514/44; 536/23.1

(57) ABSTRACT

The present invention relates to decoy oligonucleotides with the nucleic acid sequence according to SEQ ID NO: 1 to 36 and their use as pharmaceutical agents.

The present invention relates to decoy oligonucleotides with the nucleic acid sequence according to SEQ ID NO: 1 to 36 and their use as pharmaceutical agents.

The transplantation of solid organs generally represents the last resort in the treatment of diseases, in which the organ to be replaced in the recipient's body is severely damaged and/or can no longer adequately fulfil its function. This occurs, for example, in the terminal stage of heart failure, but also in cases of acute or chronic renal or hepatic failure. The pancreas, lung and small intestine are transplanted routinely but less frequently than the organs mentioned. A combined transplantation of several organs is also possible. In addition to the solid organs, the cornea of the eyes and haematopoietic stem cells from the bone marrow are also transplanted.

A total of 3130 transplantations of solid organs were carried out in Germany in 2000 (Eurotransplant). The major problem in this context is still the acute rejection of the donor organ by the recipient organism. This rejection reaction (host-versus-graft reaction) is more marked, the less closely the immunological features of the donor agree with those of the recipient (lack of histocompatibility). In the presence of adequate histocompatibility, the rejection reaction can generally be suppressed with appropriate drugs (immunosuppressants); however, long-term treatment with these drugs can lead to serious side-effects. For example, transplant patients frequently develop tumours and infections as a result of their impaired immune defences. The chronic rejection of transplanted organs is also intensified. This degeneration of the arteries and arterioles supplying the transplanted organ, also referred to as vasculopathy, represents a special, accelerated form of atherosclerosis (transplant atherosclerosis), which leads successively by progressive functional impairment to the failure of the transplanted organ. If a re-transplantation is not possible (e.g. unavailability of the organ), chronic transplant rejection inevitably leads to the death of the patient.

In immunological terms, the acute rejection of transplanted organs (not to be confused with the substantially less frequent, hyper-acute rejection reaction, which is antibody-mediated) represents a type-IV hypersensitivity reaction (delayed reaction type or delayed type hypersensitivity). The antigen (generally histocompatibility antigens on the endothelial cells lining the blood vessels of the donor organ) is phagocyted by (tissue) macrophages, processed and presented to T-helper cells (CD4-positive); the sensitisation of the T-helper cells lasts for several days. On the second contact, the T-helper cells sensitised in this manner are transformed into Th1 cells. In this context, the CD154-ligand-mediated co-stimulation of the antigen-presenting cell (this expresses the corresponding CD40-receptor) plays an important role because interleukin-12 is released from the macrophages via this signal pathway. Interleukin 12 initiates the differentiation and proliferation of the T-helper cells. For their part, the Th1-cells stimulate the formation of monocytes in the bone marrow via given growth factors (e.g. granulocyte-macrophage colony-stimulating factor), recruit these with the assistance of given chemokines (e.g. macrophage migration inhibitory factor [MIF]) and activate them via the release of interferon-γ. The resulting very severe inflammatory reaction can destroy the transplanted tissue to great extent. CD8-positive cytotoxic T-cells, which destroy their target cells by cytolysis and/or by inducing programmed cell death also participate in transplant rejection. Like the CD4-positive Th1-cells, cytotoxic T-cells can only recognise their target (the foreign cell surface) through prior antigen presentation and by “arming” themselves accordingly. In this context, CD154-CD40-mediated co-stimulation is also important. According to the latest knowledge, the endothelial cells of the donor organ themselves possibly have antigen presenting and co-stimulatory properties.

Comparable with transplant rejection, but in the reverse direction is graft-versus-host disease (GvHD), which occurs in the course of allogenic bone-marrow transplantation (between genetically non-identical individuals) in approximately 40% of recipients. In the acute phase lasting up to three months, the T-cells of the donor, transferred with the stem cells, attack the host organism; the resulting, sometimes severe inflammatory reaction manifests itself by preference in the skin and less frequently in the gastrointestinal tract and in the liver. Immunosuppression, with the potentially serious side-effects already described, is therefore also indicated in these patients. Once again, endothelial cells, this time those of the recipient organism, participate in the initiation of this inflammatory reaction. Alongside acute GvHD, there is also the chronic form, which requires a more prolonged immunosuppression.

The immunosuppressants used for the prevention of acute transplant rejection generally vary in dependence upon the organ type and/or are licensed for immunosuppression only after transplantation of certain organs. A typical treatment for recipients of a heart transplant is the combination of cyclosporin A with azathioprine and cortisone. Cyclosporin is increasingly being replaced by tacrolimus and azathioprine is being replaced by mycophenolate mofetil. Cyclosporin A, rapamycin and tacrolimus inhibit the T-cell activation; azathioprine and mycophenolatmofetil are antimetabolites; and corticosteroids act in an anti-inflammatory manner by inhibiting gene expression. In spite of their undisputed therapeutic effects, life-long systemic treatment with these drugs is inevitably associated with sometimes serious side-effects. In particular, these include myelotoxicity, neurotoxicity, nephrotoxicity, metabolic disorders even including the induction of diabetes mellitus, arterial hypertension, infections and malignancy. GvHD is generally also treated with the drugs named above, frequently in combination.

Antigen-presenting cells and T-cells communicate inter alia via CD40-receptors and CD154-ligand, and this co-stimulation plays an important role, in the context of transplant rejection and also GvHD, in the Th1-cell-mediated inflammatory reaction and/or the activation of cytotoxic T-cells. Like antigen-presenting cells, endothelial cells also constitutively express the CD40 receptor. The interaction of the endothelial cells with CD154-expressing T-helper cells (naïve T-helper cells and/or activated Th1-cells) results in an increased cellular expression of chemokines and adhesion molecules. As a result, there is an increase in the recruitment and activation of circulating monocytes; these emigrate into the vascular wall and are differentiated into macrophages. Moreover, by contrast with other antigen-presenting cells, the endothelial cells release biologically active interleukin-12 exclusively after CD40 activation. Interleukin-12 is the most important factor for the differentiation of naïve T-helper cells into Th1-cells and promotes the subsequent clonal expansion (proliferation) of the Th1-cells. The intensified formation of interferon-γ by the differentiated Th1-cells stimulates not only the activity of the infiltrated macrophages, but also intensifies the expression of CD40 in the endothelial cells. A vicious circle can develop as a result, in which the endothelial cells, T-helper cells and macrophages stimulate one another, thereby maintaining the inflammatory reaction, which damages the transplant (acute transplant rejection) and or the recipient organism (GvHD).

In this context, the blockade of the CD154/CD40-mediated co-stimulation presents a promising goal for reducing and/or inhibiting acute transplant rejection. The results from animal experiments indicate that the antibody-supported neutralisation of CD154 immediately following transplantation can even produce immunotolerance. Furthermore, chronic transplant rejection (transplant atherosclerosis) is favourably therapeutically influenced by this intervention. One disadvantage of this antibody therapy is, inter alia, the danger of hypersensitivity reactions (to the antibody), above all in the case of repeated application, and the poor accessibility at least for tissue-bound antigens (e.g. T-cells which have emigrated into the wall of the blood vessel of the donor organ), because the antibodies must generally be applied through the blood and then fail at the endothelial cell barrier.

The present invention is therefore based upon the object of providing means for a prevention and/or treatment of acute and chronic transplant rejection including GvHD and the associated consequences for morbidity and mortality of the affected patients. This object is achieved by the subject matter defined in the claims.

The invention is explained in greater detail with reference to the following diagrams.

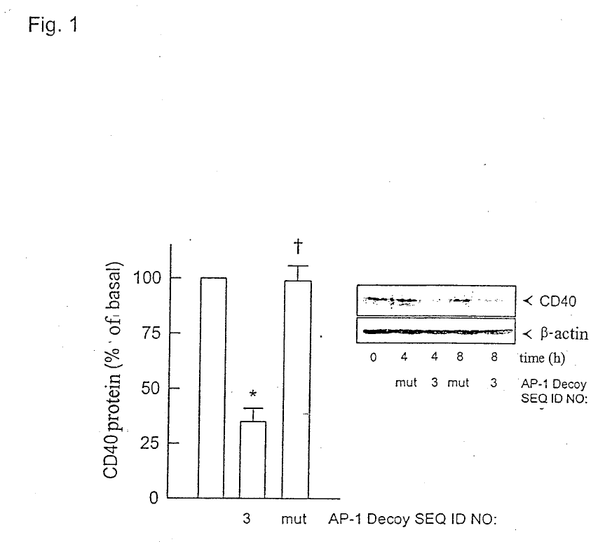

FIG. 1 shows, in the form of a bar chart, the effects of an AP-1 cis-element decoy (SEQ ID NO: 3) and a mutated control oligonucleotide (mut) on the basal CD40 protein expression in resting human endothelial cells, which were incubated for 8 hours with the corresponding oligonucleotide (10 μM) (n=7-10, statistical summary, related as a percentage to the basal expression, *P<0.05 versus basal; †P<0.05 versus cis-element decoy). The representative Western-blot analysis shows the effects (4 and/or 8 hours incubation) of the nucleic acids used on the basal CD40-protein content in resting cells. β-actin (internal standard) is used to demonstrate that identical quantities of protein were analysed.

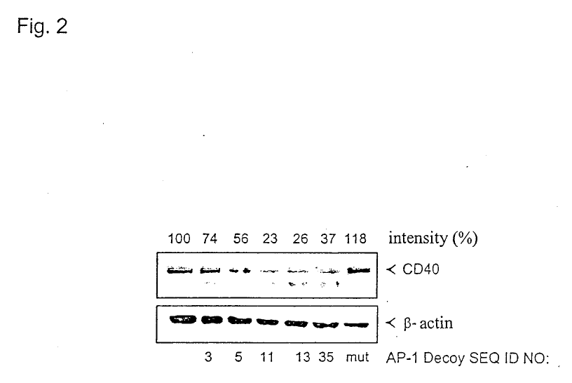

FIG. 2 shows, in a representative Western-blot analysis, the effects of selected AP-1 cis-element decoys (SEQ ID NO: 3, 5, 11, 13 and 35) and of a mutated control oligonucleotide (mut) on the basal CD40-protein expression in resting human endothelial cells, which were incubated for 8 hours with the corresponding cis-element decoys (10 μM). The relative intensities (%), measured by densiometric evaluation (One-Dscan-Gel analysis software, Scanalytics, Billerica, Mass., USA), are indicated with reference to the maximum value of the CD40-protein content in endothelial cells, which were not incubated with an AP-1 cis-element decoy.

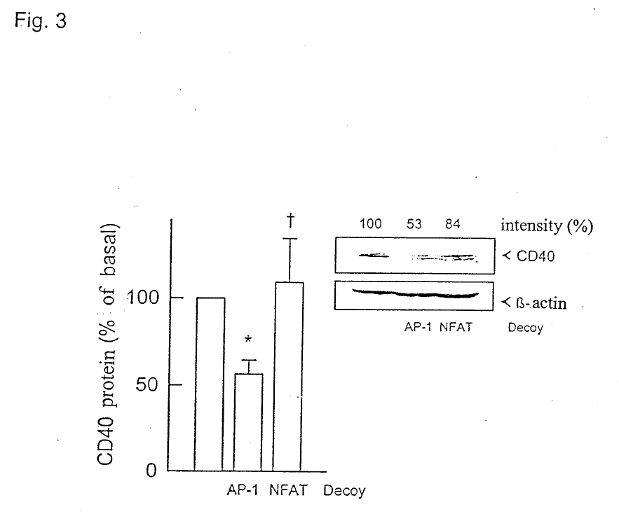

FIG. 3 shows, in the form of a bar chart and a representative Western-blot analysis with β-actin as the internal standard, the effect of the AP-1 cis-element decoy SEQ ID NO: 3 by comparison with the absence of effect of an NFAT (nuclear factor of activated T-cells) cis-element decoy on the basal CD40-protein expression in resting human endothelial cells, which were incubated for 8 hours with the corresponding oligonucleotide (10 μm). Statistical summary (n=4, related as a percentage to the basal expression; *P<0.05 versus basal; †P<0.05 versus AP-1 cis-element decoy).

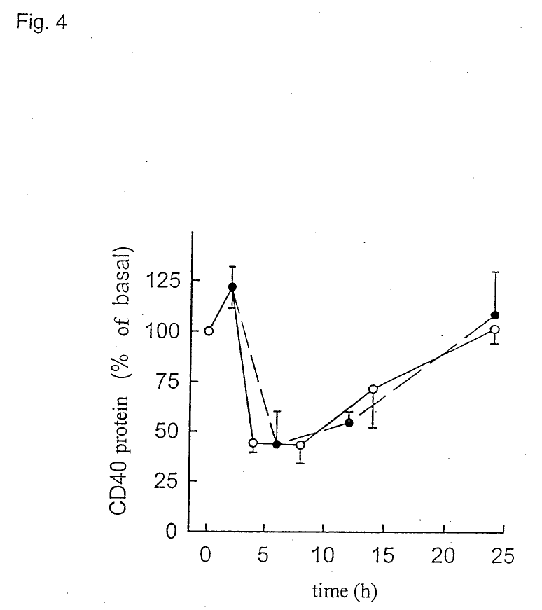

FIG. 4 shows, in the form of a linear graph, the effect of long-term exposure (empty circles) or respectively of a two-hour preliminary incubation (filled circles) with the AP-1 cis-element decoy SEQ ID NO: 3 (10 μM) on the basal CD40-protein expression in resting human endothelial cells over a period of 24 hours (n=3-7).

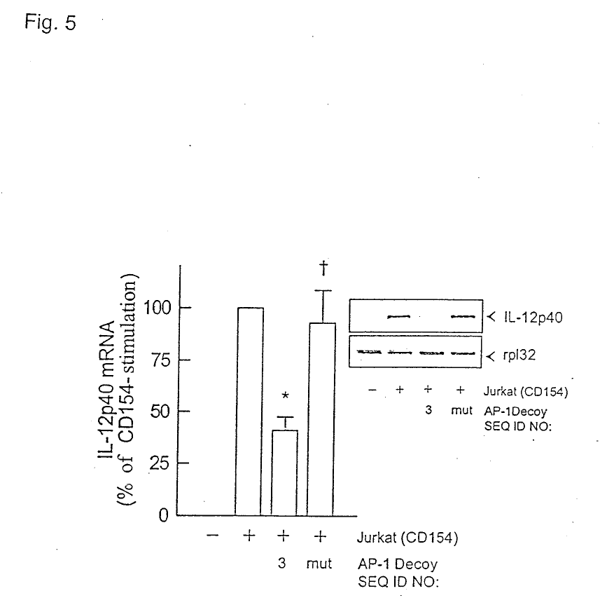

FIG. 5 shows, in the form of a bar chart and an RT-PCR analysis, the effects of a preliminary incubation (4 hours, 10 μM) with the AP-1 cis-element decoy SEQ ID NO: 3 or respectively of a mutated control oligonucleotide (mut) on the subsequent CD40 ligand-(exposure to CD40 ligand-expressing jurkat-T-cells) induced IL12-p mRNA expression in human endothelial cells (n=3). Representative RT-PCR analysis and statistical summary (related as a percentage to the maximum value of IL-12-p40 expression with CD154 stimulation, *P<0.05 versus CD154; †P<0.05 versus AP-1 cis-element decoy).

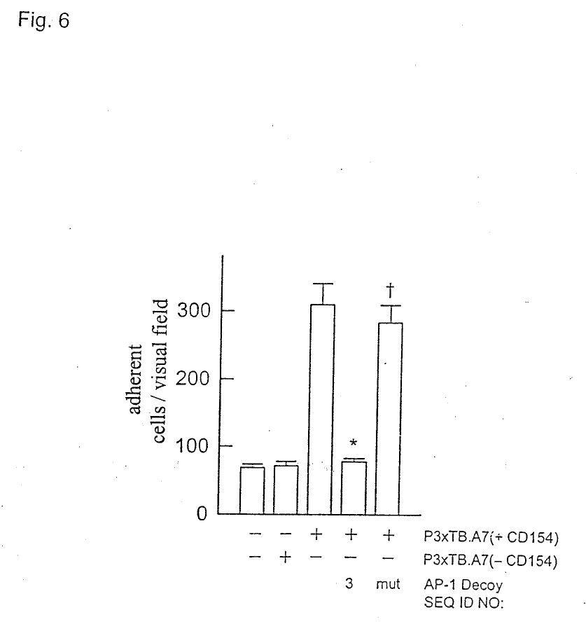

FIG. 6 shows, in the form of a bar chart, the effect of the AP-1 cis-element decoy SEQ ID NO: 3 by comparison with the control oligonucleotide (mut) on the adhesion of human THP-1 monocytes to human endothelial cells, which were pre-incubated for 8 hours with the corresponding oligonucleotide (10 μM) and then co-cultivated for 12 hours with human CD154-transfected mouse myeloma cells (P3xTB.A7, CD154) (statistical summary n=10-13, *P<0.05 versus CD154; †P<0.05 versus AP-1 cis-element decoy). Non transfected P3xTB.A7 cells (—CD154) were included as a negative control. Before the start of the THP-1-cell perfusion, the myeloma cells were almost completely removed from the endothelial cells in a washing stage with the medium.

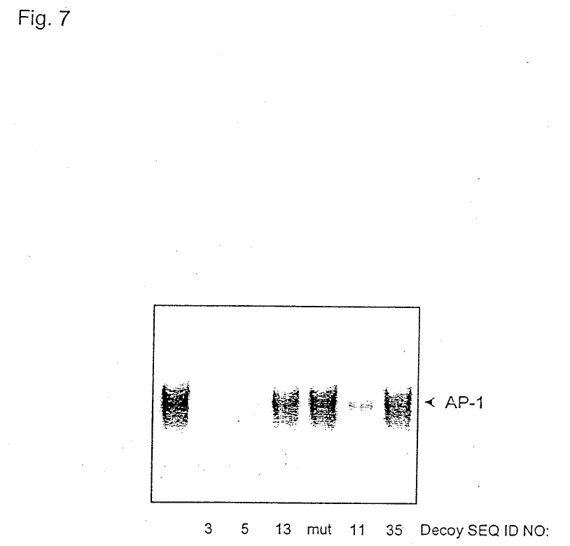

FIG. 7 shows, in a representative electrophoretic mobility-shift analysis (EMSA), the effect of a 50-fold surplus of selected AP-1 cis-element decoys (SEQ ID NO: 3, 5, 11, 13 and 35) by comparison with a mutated control oligonucleotide (mut) on the formation of DNA protein complexes between a ³²P-marked oligonucleotide (11 fmol), which binds specifically to the transcription factor AP-1, and a nuclear protein preparation from human THP-1 monocytes in a 15 μl reaction mixture.

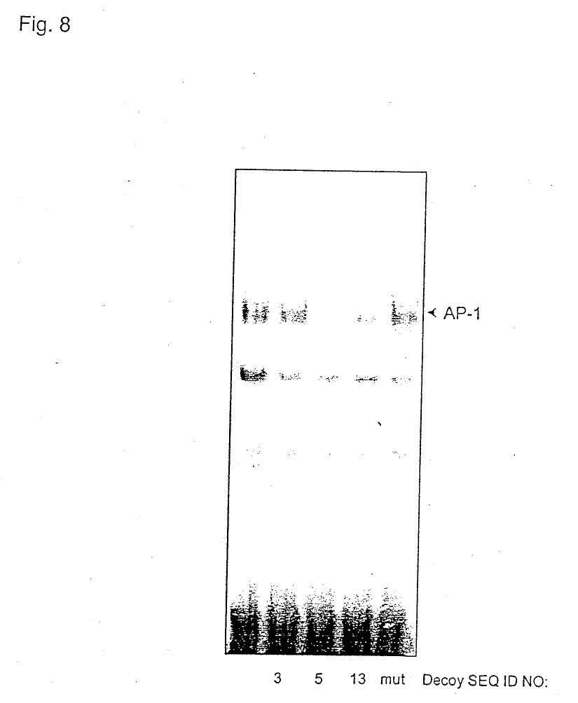

FIG. 8 shows, in a representative EMSA, the effect of selected AP-1 cis-element decoys (SEQ ID NO: 3, 5 and 13) and of a mutated control oligonucleotide (mut) on the translocation of AP-1 into the nucleus of human endothelial cells, which were incubated for 4 hours with the corresponding cis-element decoy (10 μM). Representative EMSA, which confirms the cellular absorption (and action) of the various cis-element decoys in human endothelial cells. Comparable results were obtained in at least two other independent experiments.

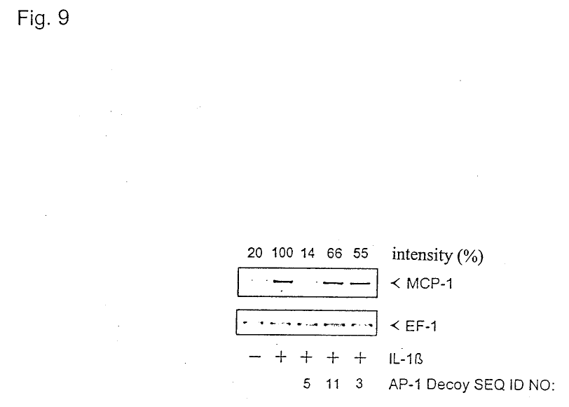

FIG. 9 shows, in a representative RT-PCR, the effects of a preliminary incubation (4 hours, 10 μM) with selected AP-1 cis-element decoys (SEQ ID NO: 3, 5 and 11) on the MCP-1 (monocyte chemoattractant protein-1) expression in human endothelial cells, which were incubated for 6 hours with 60 U/ml interleukin-1β (IL-1β). Representative RT-PCR (the relative intensities (%) measured by densiometric evaluation, are indicated relative to the maximum value with IL-1β stimulation).

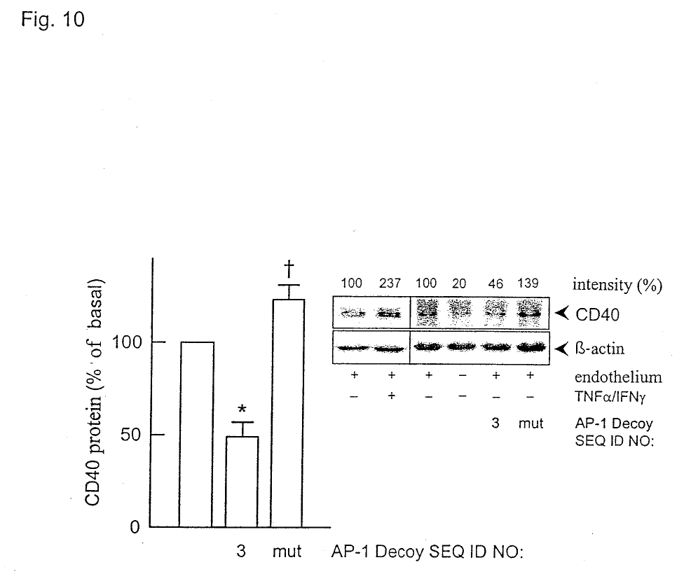

FIG. 10 shows, in the form of a bar chart and a representative Western-blot analysis, the effect of an AP-1 cis-element decoy (SEQ ID NO: 3) and a mutated control oligonucleotide (mut) on the basal CD40-protein expression in isolated endothelial-intact segments from the rat aorta, which were incubated in Waymouth medium. The cis-element decoys (10 μM) were added to the incubation medium after 1 hour pre-incubation and incubated for 11 hours with the vascular segments (6-8 segments from 5 different animals; statistical summary, related as a percentage to the basal expression, *P<0.05 versus basal; †P<0.05 versus cis-element decoy). The representative Western-blot analysis shows, by way of example, that the CD40-protein in the vascular segments investigated is primarily localised in the endothelial cells and that its expression can be significantly increased by adding the cytokine tumour necrosis factor-α (TNF-α, 1000 U/ml) and interferon-γ (IFNγ, 100 U/ml) to the incubation medium for 12 hours. The detection of β-actin (internal standard) is used to demonstrate that identical quantities of protein were analysed.

The terms “decoy oligonucleotide” and “cis-element decoy” as used in the present document refer to a double-strand DNA molecule, which provides a sequence, to which the transcription factor AP-1 binds in the cell, and which corresponds to or resembles the natural AP-1 core-binding sequence in the genome (derivative). The cis-element decoy therefore acts as a molecule for the competitive inhibition (neutralisation) of AP-1.

Transcription factors are DNA-binding proteins, which are deposited in the cell nucleus on the promoter region of one or more genes and therefore control their expression; that is to say, the new formation of the proteins, for which this gene codes. Alongside the physiologically important control of development and differentiation processes in the human body, transcription factors have a major pathogenic potential, primarily if they activate gene expression at the wrong time. Additionally, (under some circumstances, the same) transcription factors can block genes with a protective function and therefore act in a predisposing manner for the formation of a disease.

The present invention therefore consists in the provision of a decoy oligonucleotide, which is capable of binding in a sequence-specific manner to the transcription factor activator or activating protein-1 (AP-1) and which has one of the following sequences. Only one strand of the decoy oligonucleotide is shown here, but the complementary strand is also included:

| | |
|---|---|
| 5′-VTGAGTCAS-3′, | (SEQ ID NO:1) |
| where V = A, | |
| C or G and S = | |
| C or G | |
| 5′-STGACTCAB-3′, | (SEQ ID NO:2) |
| where S = C | |
| or G and B = | |
| G, C or T | |
| 5′-CGCTTGATGACTCAGCCGGAA-3′, | (SEQ ID NO:3) |
| 5′-GTGCTGACTCAGCAC-3′, | (SEQ ID NO:5) |
| 5′-GTGGTGACTCACCAC-3′, | (SEQ ID NO:7) |
| 5′-AGTGGTGACTCACCACT-3′, | (SEQ ID NO:9) |
| 5′-TGTGCTGACTCAGCACA-3′, | (SEQ ID NO:11) |
| 5′-TTGTGCTGACTCAGCACAA-3′, | (SEQ ID NO:13) |
| 5′-TGGTGAGTCACCA-3′, | (SEQ ID NO:15) |
| 5′-ATGGTGAGTCACCAT-3′, | (SEQ ID NO:17) |
| 5′-TATGGTGAGTCACCATA-3′, | (SEQ ID NO:19) |
| 5′-CTATGGTGAGTCACCATAG-3′, | (SEQ ID NO:21) |
| 5′-CCTATGGTGAGTCACCATAGG-3′, | (SEQ ID NO:23) |
| 5′-TGTTGAGTCACCA-3′, | (SEQ ID NO:25) |
| 5′-GTGTTGAGTCACCAC-3′, | (SEQ ID NO:27) |
| 5′-TGTGTTGAGTCACCACA-3′, | (SEQ ID NO:29) |
| 15′-CTGTGTTGAGTCACCACAG-3′, | (SEQ ID NO:31) |
| 5′-ACTGTGTTGAGTCACCACAGT-3′, | (SEQ ID NO:33) |
| 5′-GTCGCTTAGTGACTAAGCGAC-3′, | (SEQ ID NO:35) |

The inventors surprisingly discovered that neutralisation of the transcription factor AP-1 using corresponding decoy oligonucleotides leads within a few hours to a decline in CD40-expression in human cultivated endothelial cells (FIGS. 1-4) and also in native rat endothelial cells (FIG. 10). This effect occurred after approximately 4 hours and endured for at least 10 hours (FIGS. 1 and 4). It was also unexpected and surprising that this effect became apparent almost simultaneously and to largely the same extent at the mRNA and protein levels. However, decoy oligonucleotides, which are directed against other transcription factors (e.g. nuclear factor of activated T-cells, NFAT), do not influence the constitutive CD40 expression (FIG. 3). Control oligonucleotides, which provide an identical sequence to the AP-1 consensus core binding sequence (SEQ ID NO: 1 and 2) apart from one or two bases, also did not show this effect (FIGS. 1 and 2). Furthermore, a two-hour exposure of the endothelial cells to the AP-1 decoy oligonucleotide was adequate to suppress the CD40 expression (FIG. 4).

One consequence of the AP-1-decoy-oligonucleotide-mediated reduction of the CD40 protein content in the endothelial cells was a marked inhibition of the CD154-induced new formation of interleukin-12 p40 (FIG. 5), the rate-determining step in the synthesis of biologically active interleukin-12 (Lienenlüke et al. (2000) Eur. J. Immunol. 30, 2864). The CD154-induced expression of the vascular cell adhesion molecule-1 (VCAM-1) was also reduced to a comparable extent (58% inhibition), and in agreement with this, the CD154-induced intensified (and primarily VCAM-1 mediated) adhesion of THP-monocytes to the endothelial cells (FIG. 6).

AP-1 (http://www.cbil.upenn.edu/cgi-bin/tess/tess33?request=FCT-DBRTRV-Accno&key=T00029) is among the group, comprising approximately 46 members, of the so-called basic region leucine zipper or bZIP transcription factors. The active transcription factor generally consists of a jun/jun-homodimer or a jun/fos-heterodimer. Both fos (Genbank Accession Number V01512) and also jun (GenBank Accession Number J04111) must, for this purpose, be phosphorylated via corresponding protein kinases within the context of the cell activation, wherein the activation of this fos-kinase or respectively jun-kinase once again depends upon the activity of other protein kinases disposed higher in the signal transduction pathway (e.g. protein kinase C or stress-activated protein kinase, SEK-1). Generally in conjunction with other transcription factors, AP-1 plays an important role in the expression of a plurality of immuno-relevant genes such as interleukin-2, interleukin-4, interleukin-8, interferon-γ, MCP-1, MIF and tumour necrosis factor-α. A blockade of the activity of AP-1 can therefore interfere, for example, with the interleukin-2-dependent autocrine stimulation of T-cells and their clonal expansion.

To avoid a general weakening of the specific (cellular or respectively humoral) immune defences, the decoy oligonucleotide according to the invention is therefore preferably applied locally rather than systemically. The ex vivo treatment of a donor organ or a bone marrow donation before the transplantation represent preferred indications.

In this context, it is particularly important that the decoy oligonucleotides according to the invention become active immediately after absorption into the target cells; by contrast, the efficacy of antisense or RNA-interference oligonucleotides is primarily dependent upon the conversion of the protein in the cell and therefore upon its re-synthesis.

By contrast with the use of a corresponding control decoy oligonucleotide, if a decoy oligonucleotide according to the invention is used against AP-1 in human endothelial cells, the expression of CD40 is significantly reduced by more than 50%. Moreover, switching off the AP-1 activity leads to a highly significant inhibition of the CD154-stimulated expression of interleukin-12 p40 or respectively VCAM-1. This leads, within the context of transplant rejection or respectively GvHD, to a significant weakening of the endothelial T-cell interaction or respectively endothelial-monocyte interaction, but also of the T-cell interaction with other antigen-presenting cells (macrophages, dendritic cells and B-lymphocytes).

These effects of a decoy oligonucleotide against AP-1 (SEQ ID NO: 3) were unambiguously confirmed in a model of acute transplant rejection (heterotopic heart transplantation in rats). However, the corresponding mutated control oligonucleotide (SEQ ID NO: 47) showed no therapeutic effect and therefore illustrated the specificity of this therapeutic approach. The more than 60% inhibition of chronic transplant rejection in the same animal-experiment model by short-term exposure of the coronary arteries of the transplant to AP-1 cis-element decoy before the implantation was, on this scale, even more impressive and unexpected.

Accordingly, the use of the double-strand DNA oligonucleotides according to the invention, also referred to as decoy oligonucleotides or cis-element decoys, which contain a consensus core-binding position for AP-1, represents the preferred method for specific inhibition of AP-1 activity. The exogenous supply of a large number of transcription-factor binding positions to a cell, especially in a considerably larger number than present in the genome, produces a situation, in which the majority of a given transcription factor binds specifically to the relevant cis-element decoy and not to its endogenous target-binding positions. This approach to inhibiting the binding of transcription factors to their endogenous binding position is also referred to as squelching. Squelching (or neutralisation) of transcription factors using cis-element decoys has been successfully used to inhibit the growth of cells. In this context, DNA fragments were used, which contained specific binding positions for the transcription factor E2F (Morishita et al., PNAS, (1995) 92, 5855).

The sequence of nucleic acids, which is used to prevent the binding of the transcription factor AP-1, is, for example, the sequence, which binds naturally to the AP-1 in the cell. AP-1 binds specifically to the motif with the sequence 5′-VTGAGTCAS-3′ (SEQ ID NO:1), where V=A, C or G and S═C or G. An effective binding of AP-1 depends upon the exact agreement with this sequence, wherein the complementary strand 5′-STGACTCAB-3′ (SEQ ID NO:2), where S═C or G and B=G, C or T, can bind the transcription factor equally efficiently. The cis-element decoy can also be larger than the 9-mer core-binding sequence and can be extended at the 5′ end and/or at the 3′ end. Corresponding mutations in the region of the core-binding sequence (e.g. 5′-VTGACTCAA-3′ or 5′VTTACTTAG-3′) lead to a partial or complete loss of the binding of AP-1 to the decoy oligonucleotide (FIG. 7).

Moreover, a largely palindromic sequence of the two DNA strands favours transport into the target cells without auxiliary agents. Apart from the general requirement that the decoy oligonucleotides according to the invention effectively neutralise the transcription factor AP-1 in vitro, it is critical for therapeutic efficacy that the DNA molecule is absorbed rapidly and to an adequate extent into the target cell. This is visualised by the differential effect of neutralisation of AP-1 by the same decoy oligonucleotides in a cell-free system (FIG. 7) by comparison with intact cells (FIG. 8) or respectively their effect on gene expression in these cells (inhibition of the IL-1β stimulated expression of MCP-1; FIG. 9). Moreover, the cis-element decoy according to the invention should not exceed a given length, because this has a limiting effect on the transport into the target cell. Every decoy oligonucleotide with a length of at least 9 bp (consensus core binding sequence) up to a length of approximately 45 bp is suitable, preferably up to a length of 27 base pairs, by particular preference up to a length of approximately 23 base pairs, by particular preference with a length of 9, 10, 11, 12, 13, 14, 15, 16, 17, 18, 19, 20, 21, 22 or 23 base pairs.

Since the cis-element decoy is a double-strand nucleic acid, each DNA oligonucleotide according to the invention comprises not only the sense or forward sequence but also the complementary antisense or reverse sequence. Preferred DNA oligonucleotides according to the invention have a 9-mer core-binding sequence for AP-1, as contained in SEQ ID NO: 1. However, the cis-element decoy can also have a sequence different from the above sequence and can be longer than a 9-mer. Sequences as contained in SEQ ID NO: 3 to SEQ ID NO: 36 are particularly preferred. This listing of the preferred sequences is not finite. It is evident to a person skilled in the art that a plurality of sequences can be used as inhibitors for AP-1, so long as they fulfil the conditions of the 9-mer consensus core-binding sequence listed above and have an affinity for AP-1.

The affinity of the binding of a nucleic acid sequence to AP-1 can be determined by Electrophoretic Mobility Shift Assay (EMSA) (Sambrook et al. (1989) Molecular Cloning. Cold Spring Harbor Laboratory Press; Krzesz et al. (1999) FEBS Lett. 453, 191). This test system is suitable for quality control of nucleic acids which are intended for use in the method according to the present invention, or for determining the optimum length of a binding position. It is also suitable for the identification of other sequences which are bound by AP-1.

The method of the present invention modulates the transcription of a gene or genes in such a manner that the gene or genes, e.g. CD40, are expressed to a reduced extent or not at all. Reduced or suppressed expression within the context of the present invention means that the transcription rate is reduced by comparison which cells which have not been treated with a decoy oligonucleotide according to the invention. A reduction of this kind can be detected, for example, by Northern-blot (Sambrook et al., 1989) or RT-PCR (Sambrook et al., 1989). A reduction of this kind is typically a 2-fold, especially at least a 5-fold, in particular, at least a 10-fold reduction.

The loss of activation can be achieved, for example, if AP-1 acts with a given gene as a transcription activator, and accordingly, squelching of the activator leads to the loss of expression of the target gene. However, an indirect inhibition is also possible by modulating the mRNA-(post-transcriptional effect) or protein instability (post-translational effect) of the target gene. In this case, the neutralisation of AP-1 would lead, for example, to a reduced expression of proteins which prevent the breakdown of the mRNA of the target gene by RNases and/or which prevent the proteolytic degradation of the target protein. An effect of this kind seems to participate in the effect described above of the AP-1 cis-element decoys on the CD40-expression in the human endothelial cells.

Oligonucleotides are generally rapidly broken down by endonucleases and exonucleases, in particular, DNases and RNases in the cell. Accordingly, the DNA oligonucleotides can be modified in order to stabilise them against degradation, so that, a high concentration of the oligonucleotides is maintained within the cell over a relatively long period. Typically, a stabilisation of this kind can be obtained by the introduction of one or more modified internucleotide bonds.

A successfully stabilised DNA oligonucleotide does not necessarily contain a modification at every internucleotide bond. The internucleotide bonds at each end of both oligonucleotides of the cis-element decoy are preferably modified. In this context, the last six, five, four, three, two, the last one, or one or more internucleotide bonds within the last six internucleotide bonds can be modified. Furthermore, various modifications of the internucleotide bonds can be introduced into the nucleic acids, and the resulting decoy oligonucleotides can be tested for sequence-specific bonding to AP-1 using the routine EMSA test system. This test system allows the determination of a binding constant of the cis-element decoy and therefore allows a determination of whether the affinity has been changed by the modification. Modified cis-element decoys, which still show an adequate binding, can be selected, an adequate binding being at least approximately 50% or at least approximately 75%, and by particular preference approximately 100% of the binding of the un-modified nucleic acid.

Cis-element decoys with modified internucleotide bonds, which still show adequate binding, can be checked to determine whether they are more stable in the cell than un-modified cis-element decoys. The cells “transfected” with the cis-element decoys according to the invention are investigated at different times for the quantity of cis-element decoy still present. In this context, a cis-element decoy marked with a fluorescence-dye (e.g. Texas-Red) or a radioactively marked (e.g. ³²P or ³⁵S) cis-element decoy is preferably used with subsequent digital-fluorescence microscopy and/or autoradiography or scintigraphy. A successfully modified cis-element decoy has a half-life in the cell, which is greater than that of an un-modified cis-element decoy, preferably of at least approximately 48 hours, by greater preference at least approximately 4 days, by greatest preference at least approximately 7 days.

Suitable modified internucleotide bonds are summarised in Uhlmann and Peyman ((1990) Chem. Rev. 90, 544). Modified internucleotide phosphate residues and/or a non-phosphorus bridges in a nucleic acid, which can be used in a method according to the present invention, contain, for example, methylphosphonate, phosphorothioate, phosphorodithioate, phosphoroamidate, phosphate ester; while non-phosphorus internucleotide analogues contain, for example, siloxane bridges, carbonate bridges, carboxymethylester bridges, acetamidate bridges and/or thioether bridges. When using phosphorothioate-modified internucleotide bonds, these should preferably not be disposed between the bases cytosine and guanine, because this can lead to an activation of the target cells of the cis-element decoy.

One further embodiment of the invention is the stabilisation of nucleic acids through the introduction of structural features into the nucleic acids, which increase the half-life of the nucleic acid. Structures of this kind, which contain hairpin and dumbbell DNA, are disclosed in U.S. Pat. No. 5,683,985. At the same time, modified internucleotide phosphate residues and/or non-phosphorus bridges can be introduced together with the structures named. The resulting nucleic acids can be tested for binding and stability in the test system described above.

A cis-element decoy of the present invention is absorbed rapidly into the cell. An adequate absorption is characterised by the modulation of the expression of one or more genes, which are subject to control by AP-1 (e.g. CD40). The cis-element decoy of the present invention preferably modulates the transcription of a gene or genes after approximately 4 hours of contact with the cell, by greater preference after approximately 2 hours, after approximately 1 hour, after approximately 30 minutes and by the greatest preference after approximately 10 minutes. A typical mixture which is used in an experiment of this kind, contains 10 μmol/l cis-element decoy.

Furthermore, the present invention relates to the use of the decoy oligonucleotides according to the invention for the manufacture of a pharmaceutical agent, especially for the prevention and/or treatment of acute and chronic transplant rejection, acute and chronic graft-versus-host disease (GvHD) and ischaemic/reperfusion damage to organs following a surgical intervention.

Moreover, the invention relates to a method for modulating the transcription of at least one gene in cells, especially in endothelial cells and antigen-presenting cells (monocytes, macrophages, dendritic cells, B-cells), wherein the method comprises the step of bringing the named cells into contact with a mixture containing one or more double-strand nucleic acid(s) according to the invention, which are capable of binding in a sequence-specific manner to the transcription factor AP-1. One preferred method is, for example, the ex vivo treatment of a donor organ before introducing it into the body of the recipient by applying the nucleic-acid-containing mixture into the blood vessels of the donor organ (orthograde or retrograde).

The mixture containing the cis-element decoys according to the invention is brought into contact with the target cells (e.g. endothelial cells, epithelial cells, leukocytes, smooth muscle cells, keratinocytes or fibroblasts). The purpose of this contacting is to transfer the cis-element decoys, which bind AP-1, into the target cell (for example, the AP-1-dependent CD40-expressing cell). Accordingly, nucleic acid modification and/or additives or auxiliary substances, of which it is known that they increase the penetration of membranes, can be used within the framework of the present invention (Uhlmann and Peyman (1990) Chem. Rev. 90, 544).

In one preferred embodiment, a mixture according to the invention contains essentially only nucleic acid and buffer. An appropriate concentration of the cis-element decoy is within the range of at least 0.1 to 100 μM, preferably approximately 10 μM, wherein one or more suitable buffers can be added. An example of a suitable buffer is a modified Ringer's solution containing 145 mmol/l Na⁺, 5 mmol/l K⁺, 50 mmol/l Cl⁻, 2 mmol/l Ca²⁺, 1 mmol/l Mg²⁺, 10 mmol/l Hepes, 106 mmol/l isethionate, 10 mmol/l D-glucose, pH 7.4.

In a further embodiment of the invention, the mixture additionally contains at least one additive and/or auxiliary agent. Additives and/or auxiliary agents such as lipids, cationic lipids, polymers, liposomes, nanoparticles, nucleic acid-aptamers, peptides and proteins, which are bound to DNA, or synthetic peptide-DNA molecules are intended, for example, to increase the introduction of nucleic acids into the cell, to direct the mixture towards only one sub-group of cells, to prevent the breakdown of the nucleic acid in the cell, to facilitate the storage of the nucleic acid mixture before use. Examples of peptides and proteins or synthetic peptide-DNA molecules are, for example, antibodies, antibody fragments, ligands, adhesion molecules, all of which can be modified or un-modified.

Additives, which stabilise the cis-element decoys in the cell are, for example, nucleic-acid-condensing substances, such as cationic polymers, poly-L-lysine or polyethylenimine.

The mixture, which is used in the method of the present invention, is preferably applied locally by injection, infusion, catheter, pluronic gels, polymers, which provide a prolonged release of medicines, or any other device, which allows local access. The ex vivo application of the mixture (infusion and/or incubation) used in the method of the present invention, also allows local access.

The following drawings and examples are provided only by way of explanation and in no sense restrict the scope of the invention.

### 1. Cell Culture

Human endothelial cells were isolated from umbilical veins by treatment with 1.6 U/ml dispase in Hepes-modified tyrode solution for 30 minutes at 37° C. and cultivated on gelatine-coated 6-well tissue-culture dishes (2 mg/ml gelatine in 0.1 M HCl for 30 minutes at room temperature) in 1.5 ml M199 medium (Gibco Life Technologies, Karlsruhe, Germany), containing 20% foetal calf serum, 50 U/ml penicillin, 50 μg/ml streptomycin, 10 U/ml nystatin, 5 mM HEPES and 5 mM TES, 1 μg/ml heparin and 40 μg/ml endothelial growth factor. They were identified by their typical pavement morphology, positive immuno-staining for von Willebrandt factor (vWF) and fluorimetric detection (FACS) of PECAM-1 (CD31) and negative immuno-staining for smooth muscular α-actin (Krzesz et al. (1999) FEBS Lett. 453, 191).

The human monocyte cell line THP-1 (ATCC TIB 202), the human jurkat cell line D1.1 (ATCC CRL-10915) and the mouse myeloma cell line P3xTB.A7 were cultivated in RPMI 1640 medium (Life technologies), containing 10% foetal calf serum, 50 U/ml penicillin, 50 μg/ml streptomycin and 10 U/ml nystatin.

### 2. RT-PCR Analysis

The total endothelial RNA was isolated using the Qiagen RNeasy Kit (Qiagen, Hilden, Germany), and following this, a cDNA synthesis was carried out with a maximum of 3 μg RNA and 200 U Superscript™ II reverse transcriptase (Life Technologies) in a total volume of 20 μl in accordance with the manufacturer's instructions. For the calibration of the cDNA charge, 5 μl (approximately 75 ng cDNA) of the resulting cDNA solution and the primer pair (Gibco) for the elongation factor-1 (EF-1)-PCR with 1 U Taq DNA polymerase (Gibco) were used in a total volume of 50 μl. EF-1 was used as an internal standard for the PCR. The PCR products were separated on 1.5% agarose gels containing 0.1% ethidium bromide and the intensity of the bands was measured densiometrically with a CCD camera system and the One-Dscan gel analysis software by Scanalytics (Billerica, Mass., USA), in order to adapt the volume of the cDNA in subsequent PCR analyses.

All PCR reactions were carried out individually for each primer pair in a Hybaid OmnE Thermocycler (AWG, Heidelberg, Germany). The individual PCR conditions for the cDNA of human endothelial cells were as follows: CD40 (product size 381 bp, 25 cycles, addition temperature 60° C., (forward primer) 5′-CAGAGTTCACTGAAACGGAATGCC-3′ (SEQ ID NO: 37), (reverse primer) 5′-TGCCTGCCTGTTGCACAACC-3′ (SEQ IS NO: 38); EF-1 (product size 220 bp, 22 cycles, addition temperature 55° C., (forward primer) 5′-TCTTAATCAGTGGTGGAAG-3′ (SEQ ID NO: 39), (reverse primer) 5′-TTTGGTCAAGTTGTTTCC-3′ (SEQ ID NO: 40); IL-12p40 (product size 281 bp, 30 cycles, addition temperature 62° C., (forward primer) 5′-GTACTCCACATTCCTACTTCTC-3′ (SEQ ID NO: 41), (reverse primer) 5′-TTTGGGTCTATTCCGTTGTGTC-3′ (SEQ ID NO: 42); MCP-1 (product size 330 bp, 22 cycles, addition temperature 63° C., (forward primer) 5′-GCGGATCCCCTCCAGCATGAAAGTCTCT-3′ (SEQ ID NO: 43), (reverse primer) 5′-ACGAATTCTTCTTGGGTTGTGGAGTGAG-3′ (SEQ ID NO: 44); VCAM-1 (product size 523 bp, 26 cycles, addition temperature 63° C.), (forward primer) 5′-CATGACCTGTTCCAGCGAGG-3′ (SEQ ID NO: 45), (reverse primer) 5′-CATTCACGAGGCCACCACTC-3′ (SEQ ID NO: 46).

### 3. Electrophoretic Mobility Shift Assay (EMSA)

The nuclear extracts and [³²P]-marked double-strand consensus oligonucleotides (Santa Cruz Biotechnologie, Heidelberg, Germany), non-denatured polyacrylamide gel electrophoresis, autoradiography and supershift analysis were implemented as described in Krzesz et al. (1999) FEBS Lett. 453, 191. In this context, a double-strand DNA oligonucleotide was used with the following single strand sequence (core binding sequence is underlined): AP-1,5′-CGCTTGATGACTCAGCCGGAA-3′ (SEQ ID NO: 3). For the analysis of the displacement of endogenous AP-1 in nuclear extracts of the endothelial cells by the various cis-element decoys, a ratio of 50:1 AP-1 cis-element decoy: [³²P]-marked AP-1 oligonucleotide (11 fmol)) was selected in the EMSA binding mixture.

### 4. Decoy Oligonucleotide Technique

Double-strand decoy oligonucleotides were manufactured from the complementary single-strand phosphorothioate-linked oligonucleotides (Eurogentec, Köln, Germany) as described in Krzesz et al. (1999) FEBS Lett. 453, 191. The cultivated human endothelial cells were incubated for at least 2 hours with the relevant decoy oligonucleotide in a concentration of 10 μM. Following this, the decoy-oligonucleotide-containing medium was generally replaced with fresh medium. The single-strand sequences of the oligonucleotides were as follows (underlined letters indicate phosphorothioate-linked bases):

| | |
|---|---|
| AP-1 | |
| 5′-CGCTTGATGACTCAGCCGGAA-3′ | (SEQ ID NO:3) |
| AP-1 | |
| 5′-GTGCTGACTCAGCAC-3′ | (SEQ ID NO:5) |
| AP-1 | |
| 5′-TGTGCTGACTCAGCACA-3′ | (SEQ ID NO:11) |
| AP-1 | |
| 5′-TTGTGCTGACTCAGCACAA-3′ | (SEQ ID NO:13) |
| AP-1 | |
| 5′-GTCGCTTAGTGACTAAGCGAC-3′ | (SEQ ID NO:35) |
| AP-1 mut | |
| 5′-CGCTTGATTACTTAGCCGGAA-3′ | (SEQ ID NO:47) |
| NFAT | |
| 5′-CGCCCAAAGAGGAAAATTTGTTTCATA-3′ | (SEQ ID NO:48) |

### 5. Western-Blot Analysis

The human umbilical vein endothelial cells were opened by freezing successively five times in liquid nitrogen and thawing at 37° C. Protein extracts were manufactured as described by Hecker et al. (1994) Biochem J. 299, 247. 20-30 μg protein were separated according to a standard protocol using a 10% polyacrylamide gel electrophoresis under denaturing conditions in the presence of SDS and transferred to a BioTrace™ polyvinylidene fluoride transfer membrane (Pall Corporation, Rossdorf, Germany). A polyclonal primary anti-human-CD 40 antibody by Research Diagnostics Inc., Flanders N.J., USA was used for the immunological demonstration of the CD40 protein. The protein bands were demonstrated after adding a peroxidase-linked anti-rabbit IgG (1:3000, Sigma, Deisenhofen, Germany) using the chemiluminescence method (SuperSignal Chemiluminescence Substrate; Pierce Chemical, Rockford, Ill., USA) and subsequent autoradiography (Hyperfilm™ MP, Amersham Pharmacia Biotech, Buckinghamshire, England). The application and transfer of identical protein quantities was demonstrated after “stripping” the transfer membrane (5 minutes 0.2 N NaOH, followed by 3×10 minutes washing with H₂O) by demonstrating identical protein bands of β-actin with a monoclonal primary antibody and a peroxidase-linked anti-mouse IgG (both from Sigma-Aldrich, 1:3000 dilution).

### 6. Endothelial Cell—Leukocyte Interaction

Primary cultivated human endothelial cells, grown on cover slips up to a cell density of 100%, were washed with Hepes-tyrode buffer (data in mmol/l: NaCl 137, KCl 2.7, CaCl₂ 1.4, MgCl₂ 0.25, NaH₂PO₄ 0.4, Na-Hepes 10, D-glucose 5), which contained 1.5% polyvinylpyrrolidone (PVP; Sigma-Aldrich), and applied to the base of a perfusion chamber (2.5 mm height and 260 μl volume, Warner Instrument, Hamden, Conn., USA). The chamber was attached to an Axiovert S100 TV microscope (Zeiss, Goettingen, Germany) on a heated platform (Warner Instruments) and perfused with the heated buffer (in-line solution heater, Warner Instruments) at 37° C. The shear stress produced with a pump (Ismatec, Zurich, Switzerland) was 5 dyn/cm with a shear rate of 10 s⁻¹. The endothelial cells were initially perfused for 10 minutes with Hepes-tyrode/PVP, followed by a 10-minute superfusion with 1.5×10⁶ THP-1 cells (5×10⁵ THP-1 cells/ml) in Hepes-tyrode/PVP. Following this, the perfusion chamber was rinsed with Hepes-tyrode/PVP. The documentation of the cell-cell interactions was evaluated at 20× magnification with a SPOT RT Colour-CCD camera (Diagnostic Instruments, Burroughs St. Sterling Heights, Mich., USA). Three images from different fields of view were evaluated for each test mixture using the program MetaMorph V3.0 (Universal Imaging, West Chester, Pa., USA).

### 7. Statistical Analysis

Unless otherwise indicated, all data in the diagrams are shown as a mean value±SEM of n experiments. The statistical evaluation was implemented by one-sided variance analysis (ANOVA) followed by a Dunnett Post Test. A P-value of <0.05 was taken as a statistically significant difference.

### 8. Animal Experimental Demonstration of the Decoy-Oligonucleotide Action

To demonstrate the efficacy of the decoy-oligonucleotide-based therapy developed in the present patent application, an animal experimental Proof-of-Concept study for the indication acute transplant rejection was carried out with rats (strain combination Wistar Furth onto Lewis; experimental details, see Hölschermann et al. (1999) A. J. Pathol. 154, 211). Single application of 10 μmol/l of the AP-1-decoy oligonucleotide (SEQ ID NO: 3), but not of the mutated control oligonucleotide (AP-1 mut (SEQ ID NO: 47), no difference by comparison with the control animals), in the coronary blood vessels of the heterotopic heart transplant (30 minutes incubation prior to implantation) prolonged its survival without the administration of an immunosuppressant from 6.2±0.2 to 7.6±0.4 days (n=5, P<0.05). This effect was associated with a significant weakening of the adhesion-molecule expression (e.g. VCAM-1) in the endothelium of the coronary blood vessels of the donor hearts and the infiltration of monocytes and T-cells on post operative days 1 and 3.

The single application of the AP-1 decoy oligonucleotide also proved extremely effective in the model of transplant vasculopathy (chronic rejection). By way of deviation from the previously described, acute rejection model, the recipient animals were treated intraperitoneally with the immunsuppressant cyclosporin A (5 mg per kg body weight per day) and the donor hearts were explanted after 100 days. The degree of vasculopathy in the coronary arteries was determined morphometrically in accordance with the Adams criteria and showed the following picture: isotype control (n=7), score 0.97±0.11; cyclosporin A-control (n=6), score 2.08±0.16 (P>0.001 versus isotype control); AP-1 decoy oligonucleotide (n=6), score 1.39±0.16 (P<0.01 versus cyclosporin A-control).

1. A double-stranded DNA oligonucleotide, wherein one of the two DNA strands provides a sequence according to SEQ ID NO: 5, and the complementary DNA strand provides the sequence complementary to the these.

2. The double-stranded DNA according to claim 1 dispersed in a pharmaceutical medium.

3. A method for the prevention and/or treatment of acute and chronic transplant rejection, acute and chronic graft-versus-host disease (GvHD) and ischaemia/reperfusion damage of organs following a surgical intervention in a subject comprising administering to said subject a double-stranded DNA oligonucleotide according to SEQ. ID NO: 5.


SEQUENCE LISTING (48 sequences)

SEQ ID NO: 1 - DNA, 9 bp, Artificial Sequence
  Description of Artificial Sequence  Synthetic Primer
vtgagtcas

SEQ ID NO: 2 - DNA, 9 bp, Artificial Sequence
  Description of Artificial Sequence  Synthetic Primer
stgastcab

SEQ ID NO: 3 - DNA, 21 bp, Artificial Sequence
  Description of Artificial Sequence  Synthetic Primer
cgcttgatgactcagccggaa

SEQ ID NO: 4 - DNA, 21 bp, Artificial Sequence
  Description of Artificial Sequence  Synthetic Primer
ttccggctgagtcatcaagcg

SEQ ID NO: 5 - DNA, 15 bp, Artificial Sequence
  Description of Artificial Sequence  Synthetic Primer
gtgctgactcagcac

SEQ ID NO: 6 - DNA, 15 bp, Artificial Sequence
  Description of Artificial Sequence  Synthetic Primer
gtgctgagtcagcac

SEQ ID NO: 7 - DNA, 15 bp, Artificial Sequence
  Description of Artificial Sequence  Synthetic Primer
gtggtgactcaccac

SEQ ID NO: 8 - DNA, 15 bp, Artificial Sequence
  Description of Artificial Sequence  Synthetic Primer
gtggtgagtcaccac

SEQ ID NO: 9 - DNA, 17 bp, Artificial Sequence
  Description of Artificial Sequence  Synthetic Primer
agtggtgactcaccact

SEQ ID NO: 10 - DNA, 17 bp, Artificial Sequence
  Description of Artificial Sequence  Synthetic Primer
agtggtgagtcaccact

SEQ ID NO: 11 - DNA, 17 bp, Artificial Sequence
  Description of Artificial Sequence  Synthetic Primer
tgtgctgactcagcaca

SEQ ID NO: 12 - DNA, 17 bp, Artificial Sequence
  Description of Artificial Sequence  Synthetic Primer
tgtgctgagtcagcaca

SEQ ID NO: 13 - DNA, 19 bp, Artificial Sequence
  Description of Artificial Sequence  Synthetic Primer
ttgtgctgactcagcacaa

SEQ ID NO: 14 - DNA, 19 bp, Artificial Sequence
  Description of Artificial Sequence  Synthetic Primer
ttgtgctgagtcagcacaa

SEQ ID NO: 15 - DNA, 13 bp, Artificial Sequence
  Description of Artificial Sequence  Synthetic Primer
tggtgagtcacca

SEQ ID NO: 16 - DNA, 13 bp, Artificial Sequence
  Description of Artificial Sequence  Synthetic Primer
tggtgactcacca

SEQ ID NO: 17 - DNA, 15 bp, Artificial Sequence
  Description of Artificial Sequence  Synthetic Primer
atggtgagtcaccat

SEQ ID NO: 18 - DNA, 15 bp, Artificial Sequence
  Description of Artificial Sequence  Synthetic Primer
atggtgactcaccat

SEQ ID NO: 19 - DNA, 17 bp, Artificial Sequence
  Description of Artificial Sequence  Synthetic Primer
tatggtgagtcaccata

SEQ ID NO: 20 - DNA, 17 bp, Artificial Sequence
  Description of Artificial Sequence  Synthetic Primer
tatggtgactcaccata

SEQ ID NO: 21 - DNA, 19 bp, Artificial Sequence
  Description of Artificial Sequence  Synthetic Primer
ctatggtgagtcaccatag

SEQ ID NO: 22 - DNA, 19 bp, Artificial Sequence
  Description of Artificial Sequence  Synthetic Primer
ctatggtgactcaccatag

SEQ ID NO: 23 - DNA, 21 bp, Artificial Sequence
  Description of Artificial Sequence  Synthetic Primer
cctatggtgagtcaccatagg

SEQ ID NO: 24 - DNA, 21 bp, Artificial Sequence
  Description of Artificial Sequence  Synthetic Primer
cctatggtgactcaccatagg

SEQ ID NO: 25 - DNA, 13 bp, Artificial Sequence
  Description of Artificial Sequence  Synthetic Primer
tgttgagtcacca

SEQ ID NO: 26 - DNA, 13 bp, Artificial Sequence
  Description of Artificial Sequence  Synthetic Primer
tggtgactcaaca

SEQ ID NO: 27 - DNA, 15 bp, Artificial Sequence
  Description of Artificial Sequence  Synthetic Primer
gtgttgagtcaccac

SEQ ID NO: 28 - DNA, 15 bp, Artificial Sequence
  Description of Artificial Sequence  Synthetic Primer
gtggtgactcaacac

SEQ ID NO: 29 - DNA, 17 bp, Artificial Sequence
  Description of Artificial Sequence  Synthetic Primer
tgtgttgagtcaccaca

SEQ ID NO: 30 - DNA, 17 bp, Artificial Sequence
  Description of Artificial Sequence  Synthetic Primer
tgtggtgactcaacaca

SEQ ID NO: 31 - DNA, 19 bp, Artificial Sequence
  Description of Artificial Sequence  Synthetic Primer
ctgtgttgagtcaccacag

SEQ ID NO: 32 - DNA, 19 bp, Artificial Sequence
  Description of Artificial Sequence  Synthetic Primer
ctgtggtgactcaacacag

SEQ ID NO: 33 - DNA, 21 bp, Artificial Sequence
  Description of Artificial Sequence  Synthetic Primer
actgtgttgagtcaccacagt

SEQ ID NO: 34 - DNA, 21 bp, Artificial Sequence
  Description of Artificial Sequence  Synthetic Primer
actgtggtgactcaacacagt

SEQ ID NO: 35 - DNA, 21 bp, Artificial Sequence
  Description of Artificial Sequence  Synthetic Primer
gtcgcttagtgactaagcgac

SEQ ID NO: 36 - DNA, 21 bp, Artificial Sequence
  Description of Artificial Sequence  Synthetic Primer
gtcgcttagtcactaagcgac

SEQ ID NO: 37 - DNA, 24 bp, Artificial Sequence
  Description of Artificial Sequence  Synthetic Primer
cagagttcactgaaacggaatgcc

SEQ ID NO: 38 - DNA, 20 bp, Artificial Sequence
  Description of Artificial Sequence  Synthetic Primer
tgcctgcctgttgcacaacc

SEQ ID NO: 39 - DNA, 19 bp, Artificial Sequence
  Description of Artificial Sequence  Synthetic Primer
tcttaatcagtggtggaag

SEQ ID NO: 40 - DNA, 18 bp, Artificial Sequence
  Description of Artificial Sequence  Synthetic Primer
tttggtcaagttgtttcc

SEQ ID NO: 41 - DNA, 22 bp, Artificial Sequence
  Description of Artificial Sequence  Synthetic Primer
gtactccacattcctacttctc

SEQ ID NO: 42 - DNA, 22 bp, Artificial Sequence
  Description of Artificial Sequence  Synthetic Primer
tttgggtctattccgttgtgtc

SEQ ID NO: 43 - DNA, 28 bp, Artificial Sequence
  Description of Artificial Sequence  Synthetic Primer
gcggatcccctccagcatgaaagtctct

SEQ ID NO: 44 - DNA, 28 bp, Artificial Sequence
  Description of Artificial Sequence  Synthetic Primer
acgaattcttcttgggttgtggagtgag

SEQ ID NO: 45 - DNA, 20 bp, Artificial Sequence
  Description of Artificial Sequence  Synthetic Primer
catgacctgttccagcgagg

SEQ ID NO: 46 - DNA, 20 bp, Artificial Sequence
  Description of Artificial Sequence  Synthetic Primer
cattcacgaggccaccactc

SEQ ID NO: 47 - DNA, 21 bp, Artificial Sequence
  Description of Artificial Sequence  Synthetic Primer
cgcttgattacttagccggaa

SEQ ID NO: 48 - DNA, 27 bp, Artificial Sequence
  Description of Artificial Sequence  Synthetic Primer
cgcccaaagaggaaaatttgtttcata